(19) United States
(12) Patent Application Publication
(10) Pub. No.: US 2024/0090813 A1
(43) Pub. Date: Mar. 21, 2024

(54) BUTTERFLY HINGE SUBDERMAL NEEDLE ELECTRODE

(71) Applicant: Rhythmlink International, LLC, Columbia, SC (US)

(72) Inventors: Brian Petree, West Columbia, SC (US); Samuel William Laurie, Columbia, SC (US)

(21) Appl. No.: 18/466,913

(22) Filed: Sep. 14, 2023

Related U.S. Application Data
(63) Continuation of application No. 17/570,239, filed on Jan. 6, 2022, now abandoned.
Application No. 17/570,239 is a continuation-in-part of application No. 17/077,955, filed on Oct. 22, 2020, now Pat. No. 11,566,334.
Application No. 17/077,955 is a continuation of application No. 15/738,333, filed on Dec. 20, 2017, now Pat. No. 11,136,681.

(51) Int. Cl. A61B 5/262

(52) U.S. Cl. CPC A61B 5/262; A61B 2562/0209; A61B 2562/04

(57) ABSTRACT

Disclosed are various embodiments for a butterfly hinge electrode and uses thereof. A butterfly hinge electrode may have a first and second wing positioned adjacent to one another and connected through a coupling mechanism. The butterfly hinge electrode may have a first and second electrode assembly, the first electrode assembly positioned on the first wing and the second electrode assembly positioned on the second wing. The butterfly hinge electrode may further comprise a compression assembly. Further, the butterfly hinge electrode may be inserted into a subdermal layer of a patient for neurological monitoring through the use of the first and second electrode assemblies on the first and second wings.

### CROSS-REFERENCE TO RELATED APPLICATIONS

This application relates to and claims priority to U.S. Provisional Patent Application Ser. No. 63/407,928, filed on Sep. 26, 2022, entitled Butterfly Switch Subdermal Needle Electrode, the contents of which are herein incorporated in the entirety.

### FIELD

The present invention relates to medical electrodes, in particular to systems and methods relating to subdermal needle electrodes.

### BACKGROUND

Subdermal needle electrodes are utilized in intraoperative neuromonitoring (IONM) and during procedures such as an electroencephalogram (EEG) to monitor, record, and elicit biological signals. IONM is a procedure that protects patients by continuously monitoring the central nervous system (brain, spinal cord, and nerves) during medical operations. The real time information from IONM can work to prevent neurological injury, and further, allow a medical professional to respond with accuracy to minimize long-term post-operative damage.

Subdermal needle electrodes, when used for EEG, have the ability to insert into a subdermal region of the skin, and eliminate the need to abrade the skin to achieve low impedance. Therefore, reducing the amount of time needed to apply multiple electrodes, and further improving patient care.

A common method for attaching subdermal needles includes the use of adhesives and/or tape to ensure the needle is securely inserted into a patient's skin, as well as to reduce the chance of the subdermal needle from dislodging. This method presents several issues as the needles are often exposed, and a practitioner must position them precisely on a patient with the needle exposed.

Thus, there is a long sought need to provide a rapid deployment subdermal needle electrode, with a protective housing, wherein the protective housing provides protection for the needle assemblies, reducing accidental needle sticks. Furthermore, there exists a need for “sticking” or “gripping” force that would normally be achieved by glue or tape. Therefore, the disclosure herein provides for a butterfly hinge electrode that allows for rapid deployment that does not require the addition of adhesives or other materials, as well as a functionality that aids in preventing accidental needle sticks.

### SUMMARY

In some aspects, the techniques described herein relate to a butterfly hinge electrode, including: a first and a second wing positioned adjacent to one another, each wing having a distal end and a interior end; a first and a second electrode assembly, the first electrode assembly positioned on the first wing at the distal end and the second electrode assembly positioned on the second wing at the distal end, each electrode assembly having at least one needle electrode; a coupling mechanism, the coupling mechanism coupling the first and second wings together at the interior end of each; and a compression assembly attached to the distal end of the first wing and the second wing.

In some aspects, the techniques described herein relate to a butterfly hinge electrode, further including a hinge housing structure and a hinge cavity within the housing structure in which the first and the second wing are positioned to be protected by the hinge housing.

In some aspects, the techniques described herein relate to a butterfly hinge electrode, further including a deployment state, wherein the deployment state engages the at least one needle electrode in the first and second electrode assembly into subdermal tissue.

In some aspects, the techniques described herein relate to a butterfly hinge electrode, further including a pre-deployment state, wherein the pre-deployment state the at least one needle electrode is in a retracted state and not in subdermal tissue.

In some aspects, the techniques described herein relate to a butterfly hinge electrode, further including the hinge housing having a nodule, wherein the nodule locks the first wing and the second wing in a deployment state.

In some aspects, the techniques described herein relate to a butterfly hinge electrode, further including a deployment indicator on the hinge housing that is activated by the nodule pressing a piston into the deployment indicator, wherein when the nodule engages the deployment indicator, through the piston, it changes a color of the deployment indicator.

In some aspects, the techniques described herein relate to a butterfly hinge electrode, wherein the at least one needle electrode per the electrode assembly is curved at an angle between 60-120 degrees to allow rotational movement into subdermal tissue.

In some aspects, the techniques described herein relate to a butterfly hinge electrode, wherein the coupling mechanism is included of a cam.

In some aspects, the techniques described herein relate to a butterfly hinge electrode, wherein the coupling mechanism is included of a hinge configuration.

In some aspects, the techniques described herein relate to a butterfly hinge electrode, wherein the compression assembly includes a spring.

In some aspects, the techniques described herein relate to a butterfly hinge electrode, wherein the compression assembly includes an elastic band.

In some aspects, the techniques described herein relate to a method for applying a butterfly hinge electrode to subdermal tissue, including: provisioning a butterfly hinge electrode, including a first and second wing positioned adjacent to one another and enclosed in a hinge housing, and a first and second electrode assembly with one or more needle electrodes on each electrode assembly, and a coupling mechanism, the coupling mechanism coupling the first and the second wings together, and a compression assembly; applying the butterfly hinge electrode, wherein applying includes moving the butterfly hinge electrode from pre-deployment state to a deployment state that engages the one or more needle electrodes to subdermal tissue; and monitoring electrical signals acquired by the one or more needle electrodes on the butterfly hinge electrode.

In some aspects, the techniques described herein relate to a method, further including collapsing the first and second wing to a parallel configuration, wherein collapsing moves from the pre-deployment state to the deployment state.

In some aspects, the techniques described herein relate to a method, further including extending the first and second wing to a non-parallel configuration, wherein extending moves from the deployment state to the pre-deployment state.

In some aspects, the techniques described herein relate to a method, further including locking the coupling mechanism into the hinge housing by placing a nodule on the first and second wing into a receptacle in the hinge housing.

In some aspects, the techniques described herein relate to a method, further including unlocking the coupling mechanism from the hinge housing by removing a nodule on the first and second wing from a receptacle in the hinge housing.

In some aspects, the techniques described herein relate to a method, wherein the compression assembly includes a spring.

In some aspects, the techniques described herein relate to a method, wherein the compression assembly includes an elastic band.

In some aspects, the techniques described herein relate to a method, wherein the deployment state places the one or more needle electrodes into a subdermal layer of a patient.

In some aspects, the techniques described herein relate to a method, further including changing a color of a deployment indicator on the hinge housing by a nodule on the first and second wing engaging in a recess in the hinge housing which presses a piston into the deployment indicator.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. In the drawings:

FIG. 1 is an illustration of a straight needle electrode assembly of an example butterfly hinge subdermal electrode;

FIG. 2A-D are illustrations comparing a straight needle electrode assembly and a bent needle electrode assembly of an example butterfly hinge subdermal electrode in a pre-deployment state and a deployment state;

FIG. 3 is an illustration of an integral cam for use in moving the example butterfly hinge electrode from a pre-deployment state to a deployment state;

FIG. 4A is an illustration of an example butterfly hinge electrode in a pre-deployment state;

FIG. 4B is an illustration of an example butterfly hinge electrode in a deployment state; and

FIG. 5 is an illustration of an example butterfly hinge electrode applied to a headset template.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### I. Example Use Case Scenarios

In one aspect, the following disclosure provides for a lower profile to reduce the amount of incidents causing dislodging or removal of an electrode. In other aspects, the butterfly hinge needle electrode is further protected by a housing component, the housing component may also include features such as locking nodule on the left wing and right wing, as well as streamline packaging and deployment. The attachment configuration, allows the subdermal needles on the subdermal needle assemblies on the left and right wing to serve as points of attachment and pinching or clasping movement unto a patient, preventing dislodging. Furthermore, the hinge housing may provide protection for the practitioner when handling the subdermal needles as the electrode assembly may be at least partially sheathed. In additional aspects, the compression mechanism and cam mechanism work to move the subdermal needle assemblies from a pre-deployment state—sheathed in the hinge housing to a deployment state—wherein the subdermal needles are deployed into a patient's subdermal layer.

In further aspects, the butterfly hinge subdermal needle assembly may be configured to a headset template, allowing for easy press-on or snap-on configuration of the butterfly hinge subdermal needle. In this aspect, the butterfly hinge housing may configure to the headset in pre-defined spaces or locations, while having the subdermal needles retracted in a pre-deployment state. Therefore, shielding a practitioner as they fit the headset onto a patient. Once in place, the butterfly hinge subdermal needle assemblies may be activated by moving from a pre-deployment state to a deployment state, thus allowing the subdermal needles to acquire and or deliver electrical signals to the body of a patient.

In one aspect, a key mechanism is the butterfly hinge or coupling mechanism, which may comprise a structure and a cam. The coupling mechanism couples to a left and right wing, and may further be influenced by a compression assembly. The left and right wing may have an integrated electrode assembly positioned thereon. Each electrode assembly may hold one or more needle electrodes, which may be comprised of stainless steel, and have a length of 3 to 10 mm. Further, the needle electrodes, positioned on the needle electrode assembly, may be curved from 60 to 120 degrees to allow for subdermal penetration of the needle electrodes when in a deployment state. In other aspects, the needle electrodes may be straight, and the movement from the cam drives them at a subdermal injection angle.

In one aspect, a butterfly hinge subdermal electrode is disclosed, comprising a left and a right wing. The wing features provide for leverage for easy insertion and release of the subdermal needles, as well as ergonomic aspects for practitioners when applying. Including, in one aspect, the ability for the subdermal needles to remain shielded or guarded within the housing of the assembly. In one aspect, the left and right wings are configured with the first and second electrode assembly through a coupling mechanism. The electrode assembly is a housing for the electrodes and may be integrated into a housing structure supporting a cam or hinge of the coupling mechanism that connects the left and right wing. The electrode assembly protects the electrode needles by having the subdermal needles enclosed within the housing, as well as assists in the placement of the electrode needles by having a defined rotational area of subdermal injection. In some aspects, the subdermal needle electrode assembly is integrated into the wings as a single unit, either molded onto, carved, or affixed to the wings, and in others the electrode assembly may be removed from the wings but operate in unison, either due to a gap or spring mechanism in between, and in even other examples the needle assemblies may form their own structure that is part of the butterfly hinge electrode.

In further aspects, the butterfly hinge electrode has a coupling mechanism with an integrated cam or hinge for allowing the device to move from a pre-deployment state (wherein the needles are retracted safely into a housing or into the device itself), to a deployment state (wherein the needles are protracted and would be in the subdermal layer of a patient). There may be additional states and configurations with regard to the coupling mechanism, including a locking nodule that may be in the shape of a bubble or pin, or a third or fourth state that may reduce the amount of protrusion of the needles into the patient by limiting the amount of rotation in the coupling mechanism.

### II. Referring to the Drawings

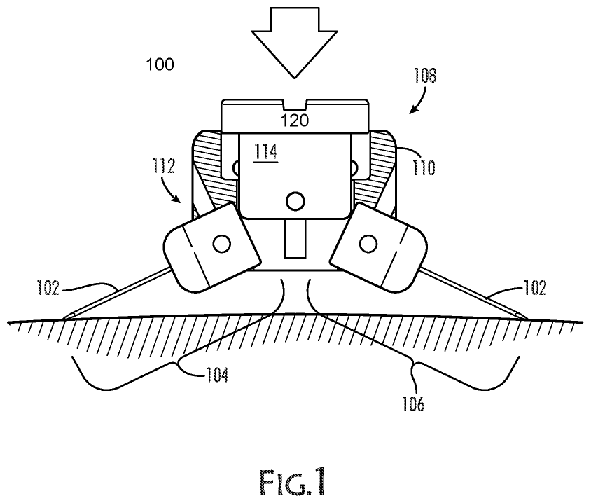

Referring now to FIG. 1, an illustration of an example butterfly hinge subdermal needle electrode (100). In one aspect, the coupling mechanism (108) is comprised of a hinge base (114) with a hinge head (120), inside a hinge housing (110), which when in a deployed state drives the coupling assembly to rotate the left and right subdermal needle assemblies (104, 106) to further inject or engage the subdermal needles (102) into subdermal tissue of a patient. A subdermal needle is a type of needle specifically designed to penetrate the skin and reach the subdermal tissue, which is the layer of tissue just below the dermis. The dermis is the middle layer of the skin, and the subdermal layer lies beneath it. Subdermal needles are typically shorter and finer compared to standard hypodermic needles used for injections into deeper tissues or veins. They are designed to minimize pain and trauma to the skin while effectively delivering medications or substances into the subdermal layer. In some aspects the butterfly hinge electrode may be comprised of an electrode assembly with a subdermal needle that is 3-10 mm in length and comprised of stainless steel. In other aspects, the subdermal needles may be curved at an angle between 60-120 degrees to allow rotational movement from the coupling assembly to drive the needles at an angle into subdermal tissue. In further aspects the subdermal needles may be substantially straight and provided for linear rotation into the subdermal tissue.

Continuing, the movement in FIG. 1 is referred to as a linear double, in which the coupling mechanism (108) drives the first and second electrode assemblies (104, 106) into place via force on the hinge head (120), acting on the hinge base (114), thereby allowing the subdermal needles (102) to pierce the tissue by rotating the first and second electrode assemblies (104, 106). In one aspect, the housing is a polymeric housing and the engagement of the subdermal needles (102) is based on pressure on the hinge head (120) and hinge base (114). The pressure drives the hinge base (114) that works to rotate the electrode assemblies (104, 106), in turn forcing a rotational movement on the subdermal needles (102). The hinge base (114), in some aspects, may have a hinge head (120) that is textured for human input and tactile feedback. In one aspect, through depression on the surface of the hinge head (120), the hinge base (114) begins moving to a deployed state, by rotating the electrode assemblies into position from the force of the hinge base (114). Furthermore, the hinge head (120) may be equipped with an ink or dye that may change colors when pressed, allowing a visual indicator for when the subdermal needle assemblies (104, 106) are engaged.

Continuing, the coupling mechanism (108) may be comprised of gaskets, and other sealed components, as well as polymeric and metal materials, but operatively allows mechanical force to act on the hinge head (120), that moves the hinge base (114) in a direction that allows the subdermal needles to retract. That movement will rotate the subdermal needle assemblies (104, 106), along with the hinge pin (112). In other aspects the left and right subdermal needle assemblies 104/106 may be termed a first and a second needle assembly, such language may be applied but the disclosure remains the same. Not shown in FIG. 1, is the connector component to the lead wire or other wires, solder, insulation and components that transmit the signals from the subdermal needles (102) to a computing device for interpretation. Further, not shown is the ability to configure the butterfly hinge subdermal needle electrode (100), into a headset or template.

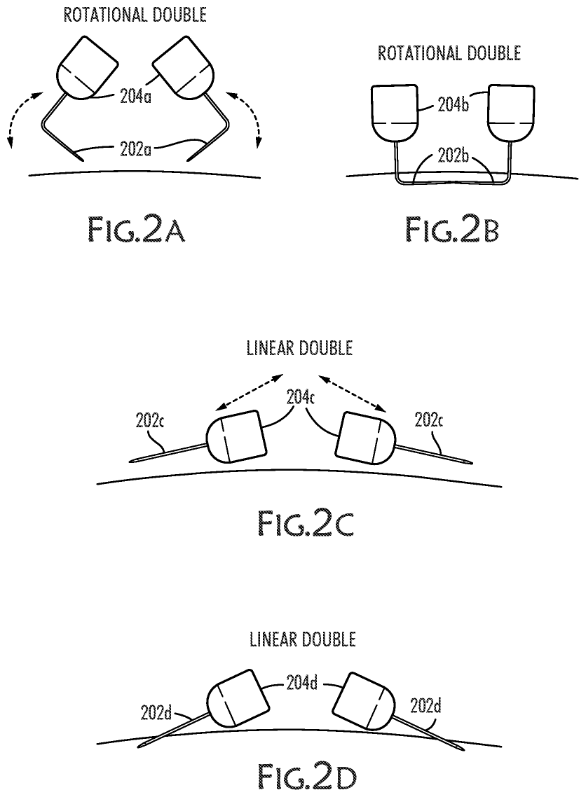

Referring now to FIGS. 2A-2D, disclosing illustrations comparing examples of a straight needle electrode assembly and a curved needle electrode assembly of a butterfly hinge subdermal electrode. In the aspects, a pre-deployment state and a deployment state are disclosed, wherein the pre-deployment state the subdermal needles are not engaged with a patient, and a deployment state the subdermal needles are engaged with a patient. In the example of FIG. 2A, a rotational double movement is disclosed in pre-deployment state, wherein curved or angled subdermal needles may be deployed to form a pincer type of movement. This pincer movement allows the subdermal needles (202a, 202b) to “stick” or form a more stable union with the patient. In the example, the subdermal needle assembly (204a, 204b) serves to both support the subdermal needle, as well as offer electrical insulation and protection. In some aspects, the housing of the subdermal needle assembly (204a, 204b) serves as the driving force from the butterfly hinge, in this regard the housing forms the coupling assembly allowing for rotation (non-geared) based on input from a hinge base.

Continuing, in FIG. 2B, the subdermal needle electrode assemblies (204b) are in a deployment state, wherein it is disclosed the angle of attack or otherwise angle of entry of the subdermal needles (202b) as a pinching or rotational movement into subdermal tissue of a patient. This particular movement allows a stable fit, and secures the subdermal needles (202b) from accidental discharge. Thus, when retracting the subdermal needles (202b), through moving from a deployed state to a pre-deployment state, the pinchers are released and the butterfly hinge is easily removed. Thus, the subdermal needle movement is mimicked in nature, as a way of clasping onto tissue and when fully deployed, preventing the needles from removal. Thus ensuring the safety of the users (nurses, physicians) as well as the patients. Further, the pinching movement also provides stability and aids in preventing the needles dislodging during an operation or scan.

Continuing, FIG. 2C is an example of a linear double, wherein the subdermal needle assemblies (204c) are deployed in an opposite direction of one another. In this aspect, the subdermal needles (202c) are in a spread position when in pre-deployment, and when deployed or in a deployment stage, rotate angularly downwards to allow subdermal needle penetration into the subdermal layer of a patient. This varies from the rotational double in that it is not a pinching movement, but provides stability and secures the subdermal needles in place by utilizing equal and opposite angles of attack.

Continuing, FIG. 2D discloses a deployed state, depicting the linear double penetrating the subdermal tissue by each subdermal needle assembly (204d) rotating downward, with the left or first assembly moving counterclockwise, and the right or second assembly moving clockwise. Thus, the engagement is opposite of one another, allowing for the force to hold the subdermal needle electrodes (202d) in place.

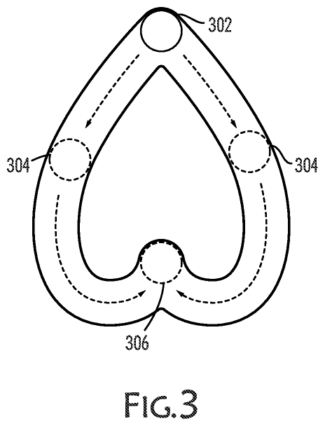

Referring now to FIG. 3, an illustration of an integral cam that may be one aspect of a coupling mechanism for use in moving the example butterfly hinge electrode from a pre-deployment state to a deployment state. In this aspect, the cam assembly may be in one of two positions, a stage one position or pre-deployment stage (302), and a stage 2 position or deployment stage (306). When not engaged, a pre-deployment state (302), the cam holds the subdermal needles within the subdermal needle assembly in a retracted position. When engaged, deployment state (306), the cam drives (304) the subdermal needle assemblies through a rotational movement that changes the relative position of the subdermal needles. In one aspect, the left and right wing is depressed, which rotates the integral cam, and drives the subdermal needles into a subdermal region of a patient, thereby allowing a practitioner to click or press on the housing to move the cam into each state.

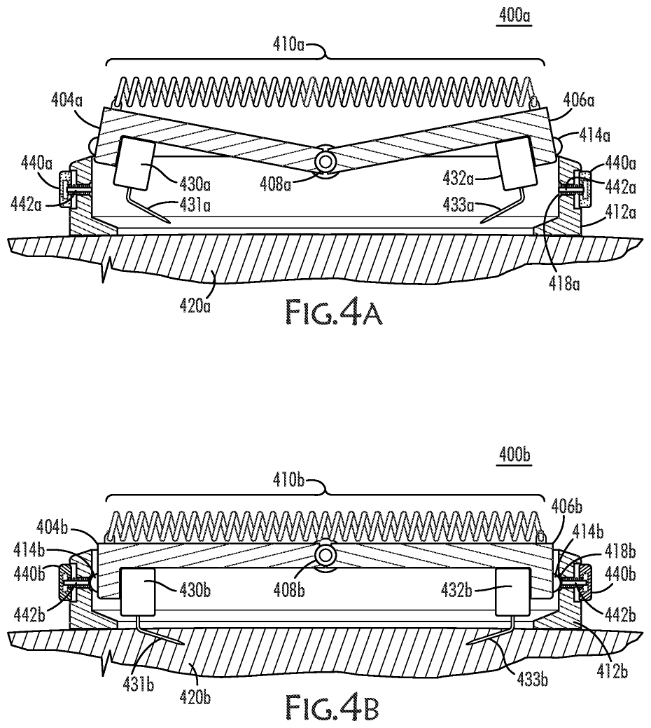

Referring now to FIGS. 4A-B, disclosing an example butterfly hinge electrode 400a in a pre-deployment state. In the first aspect, a first wing (404a) and a second wing (406a) is positioned mirrored to one another. Each wing having a distal end and a interior end, wherein the interior end is in connection with a coupling mechanism (408a, 408b). The coupling mechanism (408a, 408b) may be comprised of a mechanical connector that allows the two wings to move independent of one another. Example mechanical connectors may be a cam, a gear, a hinge, a ball joint, a clamp, or a connection that allows the first and second wing to remain in connection, but to pivot from one another, forming a rotational angle. In one aspect, a hinge if formed from the same polymeric material as the wings, and thus it forms an integrated hinge.

Continuing, on each wing there may be an electrode assembly. The first wing (404a) may have a first electrode assembly (430a) positioned at the distal end, with a subdermal needle (431a) mounted or configured therewith. Similarly, the second wing (406a) may have a second electrode assembly (432a) positioned on the second wing (406a) at the distal end, with a subdermal needle (433a) mounted or configured therewith. Each electrode assembly (430a, 432a) having at least one needle electrode, such as a subdermal needle, that is capable of configuring in a pre-deployment state, entirely within the housing (412a) and the wings (404a, 406a) of the butterfly hinge subdermal needle electrode. In one aspect, the needle electrode assemblies (430a, 432a, 430b, 432b) serve as a mounting point to anchor the subdermal needle, as well as provide insulation from electrical interference, and damage from objects. In another aspect, the needle electrode assemblies (430a, 432a, 430b, 432b) are capable of mechanical removal, thus removing the subdermal needle for upcycling and manufacture of additional butterfly hinge electrodes, without having the generate an entirely new structure.

The first and second wing (404a, 406a, 404b, 406b) may engage, and be protected with a housing structure (412a, 412b), that allows the first and second electrode assemblies to remain sheathed and protected from accidental needle stick or damage. The first and second wing (404a, 406a, 404b, 406b) may further be equipped with a nodule (414a, 414b) or raised surface at the distal end, that may be designed to engage with the housing structure (412a, 412b) to provide a haptic feedback and the ability to securely hold the hinge in a deployment state. Therefore, in one aspect, the first and second wing (404a, 406a, 404b, 406b), upon moving from a pre-deployment stage to a deployment stage, may engage the nodule (414a, 414b) with the housing (412a, 412b) to secure the wings in place and to support the clasping or pinching hold of the subdermal needles into a patient.

In one aspect, when engaging, moving from a pre-deployment state to a deployment state, or vice-versa, the subdermal electrode assemblies form a rotational double, and pinch or clasp into subdermal tissue. This pinching or grasping allows for a secure and stable connection with a patient, while also accounting for easy removal through the same rotational movement. This connection further eliminates, or greatly reduces the need for adhesives and tape to hold the electrode in place. Thus, by pinching into the tissue, it can be said the subdermal needle assembly resists horizontal and lateral movement, and prevents accidental discharge or removal. Thereby increasing patient and practitioner safety.

Continuing, the coupling mechanism (408a, 408b) couples the first and second wings (404a, 406a, 404b, 406b) together at the interior end of each wing. In one aspect, the connection through the coupling mechanism forms the butterfly hinge that provides the platform for pressing or clicking into a deployment state and/or a pre-deployment state. The coupling assembly (408a, 408b) may be any manner of physical assembly that holds the first and second wing in connection, and allows for a rotational movement around the coupling mechanism. Examples of such coupling mechanisms may be gears, hinges, cams, or other mechanical components that may be inferred from the drawings herein.

The compression assembly (410a, 410b) is attached to the distal end of the first wing (404a, 404b) and the second wing (406a, 406b) provides the opposite force to enable the first and second wing to decompress and move from a deployed state (FIG. 4B) to a pre-deployment state (FIG. 4A). The compression assembly (410a, 410b), may further be comprised of a spring configured to the respective opposite wings, or other tensioning material such as a rubber band or elastic band. The compression assembly (410a, 410b) aids in the removal and the retraction of the subdermal needle assemblies along with the subdermal needle, moving from a deployment state to a pre-deployment state. Furthermore, the compression assembly (410a, 410b) aids in preventing accidental needle stick by holding the subdermal needles within the housing. The compression assembly (410a, 410b) is held in tension by the nodules on the wings interacting with the housing, and once force is applied, allows the compression assembly to retract the subdermal needles.

Continuing, in mechanical aspects, the butterfly hinge needle electrode (400a, 400b) comprises a housing structure (412a, 412b), and a coupling mechanism (408a, 408b) cavity within the housing structure, in which the first and the second wing are positioned to be protected by the hinge housing. Thus, the housing structure (412a, 412b) forms the base that sheaths the subdermal needle assemblies (430a, 432a, 430b, 432b), including the subdermal needles, from accidental needle stick. The housing structure (412a, 412b) further serves as a locking mechanism to secure the wings when in a deployed state by receiving the nodules on the wings. In the deployment state or deployed state, the butterfly hinge engages at least one needle electrode in the first and second electrode assembly into subdermal tissue. In doing so the at least one needle electrode is capable of acquiring and delivering electrical signals to the subdermal tissue. In other aspects, there may be a plurality of needles on each subdermal needle electrode assembly.

Continuing with FIGS. 4A and 4B, illustrating example embodiments of a butterfly hinge subdermal needle electrode in a pre-deployment state and a deployment state. In further aspects, a compression assembly (410a, 410b) may be configured to the left and right wings or integrated into the cam mechanism or housing of the butterfly electrode hinge. The compression assembly (410a, 410b) assists in retracting the needles and moving from a deployment state to a pre-deployment state by applying tension on the distal ends of the wings, counteracting the coupling mechanism (408a, 408b). Any number of tensioning items may assist with the compression assembly (410a, 410b), including springs, gears, and elastic/rubber bands. In further aspects, the compression assembly is integrated with the coupling mechanism, allowing fewer parts and the ability for retraction to occur centrally and within the housing. In this aspect, the coupling assembly may be tensioned, wherein the tension releases when moved to a pre-deployment state, thereby allowing assisted removal of the grasping subdermal needles.

In further embodiments, a straight needle may be employed, wherein the coupling mechanism and/or housing may drive the straight needle into the subdermal layer. In other aspects, a bent needle may be applied, wherein a bent needle electrode from 60-120 degrees may angularly be protruded into a patient's subdermal layer. In both aspects the goal of the device remains the same, to acquire neurological signals and allow for rapid deployment by practitioners.

In further aspects, the butterfly hinge electrode (400a, 400b) may be comprised of a polymeric housing, or a metal housing, or a combination thereof (including rubber components), and may be configured to administer the needle electrodes as straight needles or bent needles. The movement of the wings and or housing may drive angular or may drive perpendicular to a patient's subdermal layer. The goals for the method of movement remains the same so long the needle pierces the subdermal layer. In further aspects, the compression assembly may be comprised of a polymeric material or of a metal, and may be integrated with the coupling mechanism. The coupling mechanism equipped to couple the left and right wing adjacent to one another, and to provide the fulcrum or amplification of transfer of force into the electrode assembly.

Referring now to FIG. 4B, in the deployment state the butterfly hinge subdermal needle electrode (400b) has a compression assembly (410b) under tension from the first wing (404b) and the second wing (406b) engaging the housing structure (414b), and the subdermal needle assemblies (430b, 432b) engaging the subdermal region of a patient's tissue. The deployment state moves the first and second wing into a parallel configuration so that the angle between the two is roughly 180 degrees. In other aspects the angle may vary depending upon the type of butterfly hinge and the properties associated with the rotational double subdermal needles.

In one aspect a spring may be utilized in the compression assembly, wherein the spring loads when the wings are tensioned apart. In another aspect an elastic band may be utilized, and in similar fashion a rubber band may be utilized. In further embodiments a torsion spring or other mechanical loading mechanism may be utilized to assist in removing the subdermal needles and returning the wings to a pre-deployment stage.

Continuing, the subdermal needles assemblies (430b, 432b) form an insulated housing, in one aspect a polymer, that supports the subdermal needle and aids in connecting the subdermal needle to a lead wire or other wire that may transmit signal to and from the subdermal tissue. Thus, the electrode assemblies may be further comprised of a connector or a connection, a lead wire, and or solder or adhesive to make the connection with the subdermal needle.

In further aspects, FIG. 4B illustrates the housing structure (412b) with a nodule receptor (442b) or locking receptor in which the nodule (416b) on the first and second wing engage to securely lock into a deployed state. Furthermore, the engagement of the nodule (416b) into the nodule receptor (442b) may compress a pin that adds pressure or force on a dye or ink, that allows an indicator (440a, 440b) to change colors or otherwise show a deployment state versus a pre-deployment state. In other aspects it may be a catch, latch, pin, tension plate, or other mechanism, or may simply be held in place by a locking assembly at the coupling mechanism. In some aspects, the coupling mechanism may integrate the compression assembly as well as a locking portion. In this aspect gears may be utilized with a ratchet system to allow the first and second wing to move from the pre-deployment state to the deployment state, as well as lock at a certain rotational point.

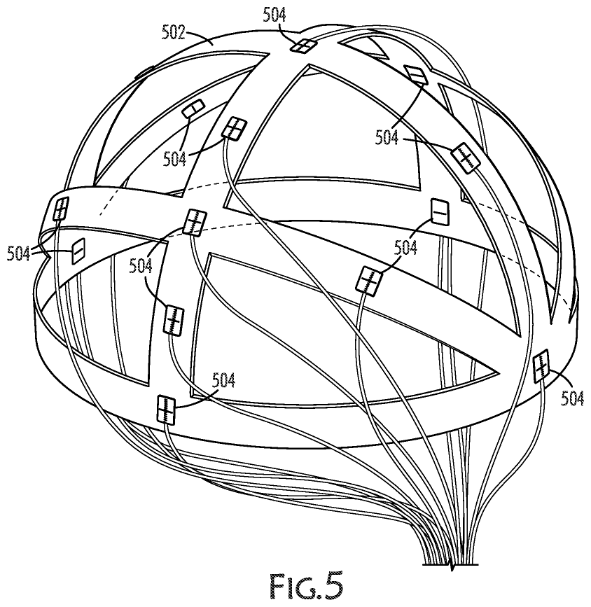

Referring now to FIG. 5, an illustration of an example butterfly hinge electrode (504) applied to a headset template (502). In one aspect a headset template (502) is defined with foramen or openings that are configured to receive the butterfly hinge electrodes (504), and that are placed at positioning relative to a patient's optimal zone's for acquiring neurological signals. In this aspect, the headset serves as a guide for placement, but also allows rapid insertion of the butterfly hinge electrode, without the usage of additional adhesives or tape.

Continuing with FIG. 5, the headset template (502) is configured with openings at specific locations that receive the butterfly hinge electrodes (504). In this aspect, a plurality of butterfly hinge electrodes is disclosed, however, in other aspects only one butterfly electrode may be applied. Further, the headset template (502) serves as a harness for the electrode wires, and allows for easy application and removal through the butterfly hinge compression assembly and coupling mechanism. In this aspect, the butterfly hinge electrodes may be pre-seated to the headset template, and thus applied by rotating the left and right wing downwards. Therefore, allowing a practitioner to rapidly lock into place utilizing the nodule on the left and right wing, allowing the needle on the subdermal needle assembly to both send and/or receive electrical signals.

It should be emphasized that the above-described embodiments of the present disclosure are merely possible examples of implementations set forth for a clear understanding of the principles of the disclosure. Many variations and modifications may be made to the above-described embodiments without departing substantially from the scope and principles of the disclosure. All such modifications and variations are intended to be included herein within the scope of this disclosure and protected by the following claims.

Therefore, the following is claimed:

1. A butterfly hinge electrode, comprising:
a first and a second wing positioned adjacent to one another, each wing having a distal end and an interior end;
a first and a second electrode assembly, the first electrode assembly positioned on the first wing at the distal end and the second electrode assembly positioned on the second wing at the distal end, each electrode assembly having at least one needle electrode;
a coupling mechanism, the coupling mechanism coupling the first and second wings together at the interior end of each; and
a compression assembly attached to the distal end of the first wing and the second wing.

2. The butterfly hinge electrode of claim 1, further comprising a hinge housing structure and a hinge cavity within the hinge housing structure, in which the first and the second wing are positioned to be protected by the hinge housing structure.

3. The butterfly hinge electrode of claim 1, further comprising a deployment state, wherein the deployment state engages the at least one needle electrode in the first and second electrode assembly into subdermal tissue.

4. The butterfly hinge electrode of claim 1, further comprising a pre-deployment state, wherein the pre-deployment state the at least one needle electrode in the first and second electrode assembly is in a retracted state and not in subdermal tissue.

5. The butterfly hinge electrode of claim 2, further comprising the hinge housing structure having a nodule receptor, wherein a nodule on the first wing and the second wing locks into the nodule receptor in a deployment state.

6. The butterfly hinge electrode of claim 5, further comprising a deployment indicator on the hinge housing structure that is activated by the nodule pressing a piston inside the nodule receptor into the deployment indicator, wherein when the nodule engages the deployment indicator, through the piston, it changes a color of the deployment indicator.

7. The butterfly hinge electrode of claim 1, wherein the at least one needle electrode per the electrode assembly is curved at an angle between 60-120 degrees to allow rotational movement into subdermal tissue.

8. The butterfly hinge electrode of claim 1, wherein the coupling mechanism is comprised of a cam.

9. The butterfly hinge electrode of claim 1, wherein the coupling mechanism is comprised of a hinge configuration.

10. The butterfly hinge electrode of claim 1, wherein the compression assembly comprises a spring.

11. The butterfly hinge electrode of claim 1, wherein the compression assembly comprises an elastic band.

12. A method for applying a butterfly hinge electrode to subdermal tissue, comprising:
provisioning a butterfly hinge electrode, comprising a first and second wing positioned adjacent to one another and enclosed in a hinge housing structure, and a first and second electrode assembly with one or more needle electrodes on each electrode assembly, and a coupling mechanism, the coupling mechanism coupling the first and the second wings together, and a compression assembly;
applying the butterfly hinge electrode, wherein applying comprises moving the butterfly hinge electrode from pre-deployment state to a deployment state that engages the one or more needle electrodes to subdermal tissue; and
monitoring electrical signals acquired by the one or more needle electrodes on the butterfly hinge electrode.

13. The method of claim 12, further comprising rotating the first and second wing to a parallel configuration, wherein rotating moves from the pre-deployment state to the deployment state.

14. The method of claim 12, further comprising extending the first and second wing to a non-parallel configuration, wherein extending moves from the deployment state to the pre-deployment state.

15. The method of claim 12, further comprising locking the coupling mechanism into the hinge housing structure by placing a nodule on the first and second wing into a nodule receptor in the hinge housing structure.

16. The method of claim 15, further comprising unlocking the coupling mechanism from the hinge housing structure by removing the nodule on the first and second wing from the nodule receptor in the hinge housing structure.

17. The method of claim 15, further comprising changing a color of a deployment indicator on the hinge housing structure, by the nodule pressing on a piston that makes contact with the deployment indicator.

18. The method of claim 12, wherein the compression assembly comprises an elastic band.

19. The method of claim 12, wherein the deployment state places the one or more needle electrodes into a subdermal layer of a patient.

20. The method of claim 12, wherein the compression assembly comprises a spring.
